(19) United States
(12) Patent Application Publication
(10) Pub. No.: US 2025/0378395 A1
(43) Pub. Date: Dec. 11, 2025

(54) AI-Based Real-Time Scheduling and Automated Rescheduling System for Diagnostic Testing Clinics

(71) Applicant: Jeniffer Scarlet Caminero, Miami, FL (US)

(72) Inventor: Jeniffer Scarlet Caminero, Miami, FL (US)

(21) Appl. No.: 19/192,893

(22) Filed: Apr. 29, 2025

(51) Int. Cl. G06Q 10/02; G16H 40/20

(52) U.S. Cl. CPC G06Q 10/027; G16H 40/20

(57) ABSTRACT

The invention provides an AI-based system for real-time scheduling and automated rescheduling of diagnostic testing appointments across a network of clinics. The system dynamically assigns appointments based on clinic availability, patient location, test urgency, and insurance compatibility. It incorporates a self-learning AI model trained on historical appointment and wait time data to optimize scheduling predictions and improve clinic throughput. A real-time monitoring module continuously tracks clinic conditions and triggers automated notifications when projected delays exceed a predefined threshold. Patients are offered options to remain at the original clinic, reschedule to a nearby clinic with shorter wait times, or cancel the appointment. The system includes a dual-view clinic interface (map and list), handles both integrated and non-integrated clinics, and ensures HIPAA-compliant handling of personal and health data. Integration with external clinic scheduling systems allows seamless data transfer and real-time synchronization. This invention significantly improves patient experience and clinic efficiency by reducing wait times and minimizing manual rescheduling efforts.

### FIELD OF THE INVENTION

This invention relates to automated appointment scheduling in healthcare, specifically for testing clinics. It utilizes artificial intelligence (AI) and real-time monitoring to dynamically assign, adjust, and optimize patient appointments, reducing wait times and improving clinic efficiency.

### BACKGROUND OF THE INVENTION

Traditional appointment scheduling systems in healthcare operate on static time slots, often leading to inefficient patient flow, long wait times, and bottlenecks when clinics experience unexpected delays. While some systems offer online appointment booking, they lack real-time adaptability and require manual intervention for rescheduling. Patients experiencing delays often must call the clinic, wait for an update, or remain onsite for extended periods.

Existing AI scheduling solutions primarily focus on provider-based scheduling (matching doctors with patients) rather than test-based scheduling (assigning patients to diagnostic testing locations). Furthermore, no current system proactively detects delays and offers real-time rescheduling at alternative clinics.

### SUMMARY OF THE INVENTION

The AI-Based Real-Time Scheduling and Automated Rescheduling System provides a dynamic and adaptive method for managing patient appointments at diagnostic testing clinics. By integrating AI-driven scheduling, real-time wait time tracking, and automated patient notifications, the system ensures that patients experience minimal wait times while clinics operate at optimal efficiency. This system introduces a self-learning AI model that continuously refines its scheduling predictions based on historical data, current demand, and patient flow trends. Additionally, when a clinic's wait time exceeds a predefined threshold (e.g., 15 minutes), the system automatically notifies affected patients via text or call, offering them the option to reschedule at a nearby clinic with a shorter wait time or cancel the appointment. Additionally, the system offers a patient-facing interface with map and list-based clinic browsing, insurance compatibility checks, real-time wait time availability, and streamlined manual options for non-integrated clinics, thereby improving access while maintaining interoperability with existing systems.

### BRIEF DESCRIPTION OF DRAWINGS

FIG. 1 is a flowchart illustrating the operation of the AI-based real-time scheduling and automated rescheduling system for diagnostic testing clinics. The process begins with patient intake and profile setup, including consent for location and insurance data access. The system then uses an AI engine to generate optimal clinic and appointment recommendations, displays options based on clinic integration status, and allows the patient to confirm an appointment. The system actively monitors clinic wait times in real time and, upon detecting delays exceeding a configured threshold (e.g., 15 minutes), notifies the patient and offers options to continue waiting, reschedule to a nearby clinic, or cancel. Based on the patient's selection, the system updates the appointment and notifies all involved clinics.

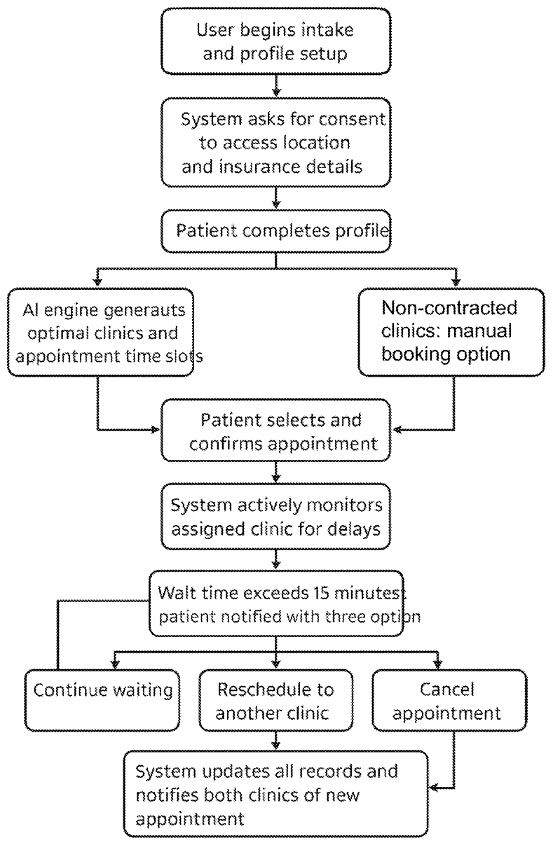

### DETAILED DESCRIPTION OF THE INVENTION

The disclosed system is a computer-implemented AI-based scheduling and automated rescheduling platform designed for diagnostic testing clinics operating across multiple locations. The system facilitates dynamic patient appointment allocation, real-time monitoring of operational conditions, and automatic rescheduling when delays are detected.

### System Modules

### 1. Patient Intake and Profile Setup Module

Guides users through initial setup by collecting:
Location access (with explicit consent via pop-up prompt)
Personal details (name, date of birth, contact info)
Insurance provider and policy information
Optional upload of physician's test order (via image upload or EHR integration)
Ensures a personalized and streamlined scheduling experience.
Stores data securely and only shares necessary details with selected clinics upon appointment confirmation.

### 2. Clinic Search and Display Interface (Dual-View UI)

Offers two modes:
List View: Presents clinics as a scrollable list with names, distances, appointment availability, wait time (if available), insurance acceptance, and booking actions.
Map View: Plots nearby clinics on a map with interactive markers that reflect real-time data and contracted status.
UI mimics familiar formats (e.g., Zillow, Uber Health), improving user adoption and experience.

### 3. Non-Integrated Clinic Display Logic

If a clinic does not share real-time data:
Displays “Wait Time Unavailable”
Disables in-app booking
Automatically presents:
Clinic hours of operation
Tap-to-call phone number
Address and instructions to bring a physician's order
Ensures the user can still proceed with minimal disruption, even if full integration is unavailable.

### 4. AI-Based Scheduling Engine

Assigns patient appointments based on:
   Real-time availability of diagnostic testing clinics,
Patient proximity, determined via GPS (with user consent) or IP-based geolocation,
   Test urgency as provided by the ordering healthcare provider.
   Incorporates insurance compatibility and clinic capacity constraints when applicable.

### 5. Real-Time Monitoring Module

Continuously tracks operational metrics including:
Patient check-ins via kiosks or reception systems,
Number of ongoing and completed tests,
Clinician or technician availability.
Calculates actual vs. scheduled start times and identifies discrepancies.

### 6. Self-Learning AI Model

The AI model uses supervised learning techniques, trained on labeled historical data such as:
Appointment creation timestamps,
Patient arrival and check-in times,
Start and end times of diagnostic procedures.
The model refines predictions for wait time duration, appointment slot optimization, and clinic throughput.

### 7. Automated Patient Notification System

Upon detection that a clinic's real-time queue duration exceeds a configurable delay threshold (e.g., 15 minutes), the system automatically triggers a patient notification process.
Patients are contacted via two-way SMS, push notification through the mobile interface, or an AI-driven automated voice call. The alert explicitly states the delay and presents the patient with three actionable options:
Remain at the current clinic and proceed with their originally scheduled appointment.
Reschedule to a nearby clinic with a shorter real-time queue.
Cancel the appointment and optionally rebook at a later time.
The system is interactive, allowing patients to confirm their choice instantly. If the patient selects rescheduling, the automated module updates the appointment in real time, transfers relevant data and test orders to the new clinic (if integrated), and sends updated notifications to both the original and new clinics.

### 8. Automated Rescheduling and Record Transfer Module

If the patient accepts rescheduling:
   The system selects a new clinic based on proximity, availability, and insurance compatibility.
   Patient data, diagnostic orders, and scheduling metadata are securely transferred to the selected clinic.
   Both the original and receiving clinics receive notification of the updated appointment.
   Ensures seamless continuity of patient care, accurate record handover, and minimal disruption.
      If the new clinic is non-integrated, a manual confirmation request is sent, and the patient is notified with instructions to bring relevant documentation.

### 9. Appointment Review & Confirmation Page

After selection, patients receive a final review screen displaying:
Clinic name, address, phone number, appointment date/time
Instructions based on integration level
Downloadable confirmation or email/text receipt
Upon confirmation:
Patient data is securely sent to the clinic
Scheduling system (if integrated) is updated in real time
Clinics receive all necessary diagnostic order info

### 10. Scheduling System Integration Module

Enables two-way communication with clinic scheduling platforms using:
Secure APIs
HL7 or FHIR interoperability standards
Serves as a middleware layer, allowing seamless read/write scheduling while preserving the clinic's existing software and workflows
Avoids vendor lock-in and supports scalability across heterogeneous clinic networks

### 11. Cloud-Based Synchronization and Admin Dashboard

The entire network of diagnostic testing clinics is integrated via a HIPAA-compliant, secure cloud infrastructure.
Real-time synchronization ensures consistency across all scheduling and communication records.
An administrative dashboard provides clinic managers and network operators with analytics on appointment flow, wait times, and load distribution.
The dashboard supports manual overrides, alert customization, and system diagnostics.

### Advantages Over Existing Systems and Patient Flow Improvements:

Proactive AI Scheduling vs. Static Booking Systems-Unlike traditional scheduling, which assigns fixed time slots, this system dynamically adjusts appointments in real time based on current conditions.

Predicts and Reduces Wait Times—Current scheduling systems only notify patients after a delay occurs. This system anticipates delays before they impact patients and offers immediate alternatives.

Minimizes Patient Frustration &amp; No-Shows—By proactively notifying and rescheduling patients, the system prevents unnecessary waiting and reduces missed appointments.

Improves Clinic Efficiency—Load-balancing appointments across multiple locations optimizes resource usage, preventing overburdened clinics while keeping others fully utilized.

Self-Learning AI for Continuous Improvement—Unlike traditional scheduling tools, which rely on fixed logic, this system continuously adapts and refines scheduling predictions based on real-world data.

### Data Privacy and Security

The system is designed in accordance with HIPAA and applicable regional healthcare data privacy laws. Patient location data is accessed only upon explicit consent via the mobile user interface and is processed securely. All data transfers are encrypted and logged to ensure auditability and compliance. All collected data adheres strictly to HIPAA and local healthcare privacy regulations. Data is encrypted both in transit and at rest. Consent is required before accessing geolocation or personal health details.

### Use Case Flow (Example Implementation)

1A. User begins intake and profile setup
1B. System asks for consent to access location and insurance details
1C. Patient completes profile
2A. Clinic search results are displayed in two views: List View and Map View
2B. Contracted clinics show wait times and allow booking
2C. Non-contracted clinics display a manual booking option with clinic hours and phone number
3. A patient accesses the scheduling user interface via mobile application or website.
4. The system retrieves:
   Real-time availability from networked diagnostic clinics,
   Patient's current location (with consent),
   Test urgency provided by the physician.
4A. Clinic List Display Based on Network Status:

If clinic is contracted with the system: show available slots, estimated wait times, and a ‘Book Now’ option.

If not contracted: show ‘Call to Schedule’, mark wait time as unavailable, and instruct the patient to bring order form.
5. AI engine generates the optimal clinics and appointment time slots.
6. Patient selects and confirms appointment.
7. System actively monitors the assigned clinic for delays.
8. If the system detects that the wait time at the selected clinic is projected to exceed 15 minutes:
   The patient is notified immediately.
   The notification presents three options:
   a) Continue waiting,
   b) Reschedule to another clinic, or
   c) Cancel appointment.
      Based on the patient's input, the system dynamically updates the appointment, data transfers, and confirmations.
9. Patient accepts a rescheduling option for a nearby clinic with a shorter wait.
10. System updates all records and notifies both clinics of the new appointment.

### Detailed Description and Non-Obvious Improvements Over Prior Art

The present invention discloses a novel AI-based scheduling and rescheduling system specifically engineered for diagnostic testing clinics. It includes a machine learning model trained via supervised methods on historical test data, patient arrivals, durations, and no-show patterns.

Unlike conventional systems, this invention leverages real-time GPS/IP-based geolocation, urgency stratification, and dynamic wait time modeling, all within HIPAA-compliant constraints. It autonomously adjusts to cancellations, delays, and emergent high-priority scheduling demands.

Patients are engaged via multi-channel communications and experience minimal friction during rescheduling. This integration of adaptive logic, predictive analytics, and privacy-aligned operations is a significant advancement over static, manual scheduling platforms.

Accordingly, the invention is not an obvious extension of prior art but represents a purpose-built healthcare logistics solution addressing a long-standing inefficiency in diagnostic clinic operations.

### Hybrid Clinic Network Integration and Display Logic

Upon receiving a zip code or geolocation signal, the system displays diagnostic clinics within a defined radius. Contracted clinics show real-time scheduling, wait times, and allow direct bookings. Non-contracted clinics are clearly labeled and provide only contact information and a reminder to bring a physician's order.

This ensures inclusive access for patients, while transparently indicating the difference in integration levels between clinics.

1. A computer-implemented system for scheduling and rescheduling diagnostic testing appointments using artificial intelligence, comprising an AI-based scheduling engine configured to assign appointments based on clinic availability and patient proximity; a monitoring module that tracks appointment status and detects delays; and a communication interface that allows rescheduling or cancellation based on clinic wait times.

2. The system of claim 1, wherein the scheduling engine assigns appointments based on real-time availability of a plurality of diagnostic testing clinics, geographic proximity of the patient determined by GPS or IP-based geolocation with consent, urgency level of the test, and compatibility with the patient's health insurance network.

3. The system of claim 2, wherein the AI model is trained using supervised learning on historical appointment data, including timestamps of patient arrivals, test durations, and no-show patterns, and refines wait time predictions and scheduling efficiency over time.

4. The system of claim 2, wherein the monitoring module triggers an alert to the patient when the projected wait time at the selected clinic exceeds a configurable threshold, and the communication interface presents the patient with options to remain, reschedule to a different clinic, or cancel the appointment.

5. The system of claim 4, wherein upon patient acceptance of rescheduling, the system automatically reassigns the appointment, transfers patient data and diagnostic orders to the new clinic, and notifies both the original and reassigned clinics of the updated status.

6. The system of claim 1, further comprising a dual-view user interface that displays diagnostic testing clinics in both a list format and an interactive map format, wherein contracted clinics allow real-time booking and non-contracted clinics display contact information and a prompt to bring test orders.

7. The system of claim 1, wherein a final appointment confirmation page displays clinic details, appointment time, patient and insurance information, and transmits said data to the clinic's scheduling software via secure API or encrypted messaging.

8. The system of claim 1, wherein all data exchanges are HIPAA-compliant and adhere to healthcare interoperability standards.
